(19) United States
(12) Patent Application Publication
(10) Pub. No.: US 2024/0210585 A1
(43) Pub. Date: Jun. 27, 2024

(54) Assessing and Monitoring Hydrocarbon Exploration Data Maturity

(71) Applicant: Saudi Arabian Oil Company, Dhahran (SA)

(72) Inventors: Ahmed A. AlSaleh, Dammam (SA); Turki A. AlGhamdi, Dhahran (SA); Sara AlBarrak, Al Khobar (SA); Ali Babiker I. Suliman, Dhahran (SA); Rami Majad Aljawad, Al Qatif (SA)

(21) Appl. No.: 18/086,256

(22) Filed: Dec. 21, 2022

(51) Int. Cl. G01V 1/30; G01V 1/36; G06F 16/23

(52) U.S. Cl. CPC G01V 1/30; G01V 1/36; G06F 16/2365

(57) ABSTRACT

A method for assessing the maturity of data includes cataloging types of the data; developing key performance indicators for each data type; developing quality, governance, availability, and security rules for each data type; updating a database storing the data; and assessing quality, governance, availability, and security of the data based on the rules for each data type.

### BACKGROUND

Hydrocarbon exploration is a high risk, high cost activity. Decisions about exploration and production strategies are based on field measurements that can have different levels of quality, geographic extent, availability, and security requirements.

### SUMMARY

This specification describes systems and methods for supporting hydrocarbon exploration and production activities based on assessing the maturity of data types. For example, data types can be classified into five major categories of wells, sub surface, surface geology, geophysical and geospatial data category. The top priority key data types identified as wells approvals, well planning, well requirements, well header (location, elevation, classification, status), directional survey, core data, well picks, checkshots, well logs, hydrocarbon shows, casing, perforations, well reports, lithology, stratigraphy, geochemistry, VSP, magnetic, gravity, seismic stacks, synthetics, inversion and geospatial datasets. These datasets can be used to develop and implement exploration and production strategies for a hydrocarbon field. This approach provides a unified data model and dashboard to measure and monitor the statuses of quality business rules, governance standards, availability and accessibility and security of the data types, although this description uses data types associated with hydrocarbon exploration and production, this approach can be applied to other data types produced by an organization.

This approach uses a scoring concept is designed to measure how good the data is based on predefined key performance indicators and business rules. In a prototype, quality, governance, availability and security (four components associated with data maturity) were scored separately, then, all four components are averaged into one data maturity score for a single data type. In the same context, the score for an organization can be measured by averaging the data maturity scores for all the data types produced by that organization. The prototype was implemented with a data maturity dashboard that reflects both the trusted data and the data that requires improvements in an organization.

In one aspect, methods for hydrocarbon exploration of a subsurface reservoir based on assessing the maturity of data on the subsurface reservoir include generating estimates and measurements of properties of the subsurface reservoir; cataloging data types of the estimates and measurements of properties of the subsurface reservoir; developing key performance indicators for each data type; developing quality, governance, availability, and security rules for each data type; updating a database storing the estimates and measurements of properties of the subsurface reservoir; assessing quality, governance, availability, and security of the estimates and measurements of properties of the subsurface reservoir based on the rules for each data type; and performing exploration activities based on field properties.

In one aspect, methods for assessing the maturity of data include: cataloging types of the data; developing key performance indicators for each data type; developing quality, governance, availability, and security rules for each data type; updating a database storing the data; and assessing quality, governance, availability, and security of the data based on the rules for each data type.

Implementations of these methods can include one or more of the following features.

In some embodiments, methods also include incorporating estimates and measurements of properties of the subsurface reservoir into the database and performing quality assurance/quality control review of the estimates and measurements of properties of the subsurface reservoir.

In some embodiments, assessing the quality, governance, availability, and security of the estimates and measurements of properties of the subsurface reservoir comprises assigning a first score representing the quality, a second score representing the governance, a third score representing the availability, and a fourth score representing the security for single data type. In some cases, methods also include averaging all rules for one data type to provide a score for that data type. In some cases, methods also include averaging all data types belonging to an organization to provide a score for that organization.

In embodiments, developing quality, governance, availability, and security rules for each data type comprises identifying at least one master repository for each data type.

This approach helps verify that each data type is governed and available to users with high quality and controlled security. In contrast to methods that measure the quality, governance or security access of data types separately, this approach provides a comprehensive way of measuring, monitoring and assessing the status of data maturity (e.g., for quality, governance, availability, and security) at the same time from one dashboard platform that reads from a single database model. This approach allows data management organizations to maintain an independently dynamic sets of data maturity metrics for each data type and business rules associated with, for example, quality, governance, availability, and security in different corporate databases and master repositories.

This approach provides a process to periodically monitor the progress of quality, governance, availability and security of each data type in a data catalog through predefined key performance indicators. Each data type is given a score between 0 and 100% for each of the components of assessment. In a prototype, these components were quality, governance, availability, and security. All four components are averaged together into one score for a single data type. Multiple data types produced by a department make up the score for that department. These scores provide a clear view on the strengths and the areas of improvements for each data type in an organization through a specific purpose dashboard.

For example, the dashboard scores can be monitored daily with weekly reports to management. This approach can aid in improving the level of data maturity in quality, governance, availability, and security by addressing the top-five data types with the highest and lowest score. The dashboards can be analyzed and investigated by stakeholders, data managers, proponents, data users, and data ambassadors who accountable for two ways communications between data managers and the data owners to improve the usefulness of data in the exploration.

The details of one or more embodiments of these systems and methods are set forth in the accompanying drawings and the description below. Other features, objects, and advantages will be apparent from the description and drawings, and from the claims.

### DESCRIPTION OF DRAWINGS

FIG. 1 is a schematic view of exploration activities being performed to characterize a subsurface formation.

FIG. 2 is a flow diagram illustrating an approach supporting hydrocarbon exploration and production activities based on assessing the maturity of data types being used to develop and implement exploration and production strategies for a hydrocarbon field.

FIGS. 3A and 3B are schematics illustrating the components of a system implementing this approach.

FIGS. 4A and 4B are screen shots of the graphic user interface of a prototype system implementing the approach of FIG. 2.

FIG. 5 is a block diagram illustrating an example computer system used to provide computational functionalities associated with described algorithms, methods, functions, processes, flows, and procedures as described in the present disclosure, according to some implementations of the present disclosure.

Like reference symbols in the various drawings indicate like elements.

### DETAILED DESCRIPTION

This specification describes systems and methods for supporting hydrocarbon exploration and production activities based on assessing the maturity of data types being used to develop and implement exploration and production strategies for a hydrocarbon field. This approach provides a unified data model and dashboard to measure and monitor the statuses of quality business rules, governance standards, availability and accessibility and security of the data types, although this description uses data types associated with hydrocarbon exploration and production, this approach can be applied to other data types produced by an organization.

This approach uses a scoring concept is designed to measure how good the data is based on predefined key performance indicators and business rules. In a prototype, quality, governance, availability and security (four components associated with data maturity) were scored separately, then, all four components are averaged into one data maturity score for a single data type. In the same context, the score for an organization can be measured by averaging the data maturity scores for all the data types produced by that organization. The prototype was implemented with a data maturity dashboard that reflects both the trusted data and the data that requires improvements in an organization.

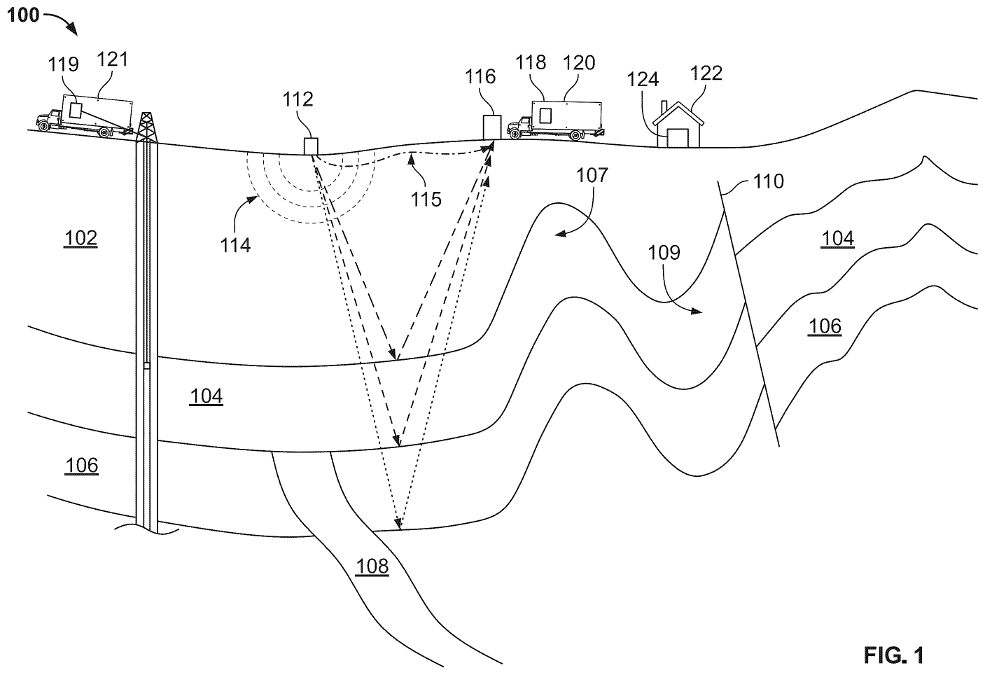

FIG. 1 is a schematic view of a seismic survey and other field activities being performed to map subsurface features such as facies and faults in a subsurface formation 100 and to generate data about the subsurface formation 100. The different data types (e.g., well planning, well requirements, well header, directional survey, formation topes, core data, checkshots, well logs, well testing, hydrocarbon shows, casing, perforations, well reports, lithology, facies, stratigraphy, geochemistry, VSP, magnetic, gravity, seismic survey, seismic stacks, faults, horizon, prospect, synthetics, inversion, fields, reservoir and geospatial datasets) have different levels of quality, governance, availability, and security requirements. For example, estimates of the location of layers of the subsurface reservoir 100 generated based on a seismic cover larger area but are less accurate than observed depths of layers based on taking core samples and performing logging in test wells. The different characteristics of the different data types lead to different criteria being used to assess the maturity of the different data types.

The subsurface formation 100 includes a layer of impermeable cap rock 102 at the surface. Facies underlying the impermeable cap rocks 102 include a sandstone layer 104, a limestone layer 106, and a sand layer 108. A fault line 110 extends across the sandstone layer 104 and the limestone layer 106.

Oil and gas tend to rise through permeable reservoir rock until further upward migration is blocked, for example, by the layer of impermeable cap rock 102. Seismic surveys attempt to identify locations where interaction between layers of the subsurface formation 100 are likely to trap oil and gas by limiting this upward migration. For example, FIG. 1 shows an anticline trap 107, where the layer of impermeable cap rock 102 has an upward convex configuration, and a fault trap 109, where the fault line 110 might allow oil and gas to flow in with clay material between the walls traps the petroleum. Other traps include salt domes and stratigraphic traps.

A seismic source 112 (for example, a seismic vibrator or an explosion) generates seismic waves that propagate in the earth. Although illustrated as a single component in FIG. 1, the source or sources 112 are typically a line or an array of sources 112. The generated seismic waves include seismic body waves 114 that travel into the ground and seismic surface waves 115 travel along the ground surface and diminish as they get further from the surface.

The velocity of these seismic waves depends on properties, for example, density, porosity, and fluid content of the medium through which the seismic waves are traveling. Different geologic bodies or layers in the earth are distinguishable because the layers have different properties and, thus, different characteristic seismic velocities. For example, in the subsurface formation 100, the velocity of seismic waves traveling through the subsurface formation 100 will be different in the sandstone layer 104, the limestone layer 106, and the sand layer 108. As the seismic body waves 114 contact interfaces between geologic bodies or layers that have different velocities, each interface reflects some of the energy of the seismic wave and refracts some of the energy of the seismic wave. Such interfaces are sometimes referred to as horizons.

The seismic body waves 114 are received by a sensor or sensors 116. Although illustrated as a single component in FIG. 1, the sensor or sensors 116 are typically a line or an array of sensors 116 that generate an output signal in response to received seismic waves including waves reflected by the horizons in the subsurface formation 100. The sensors 116 can be geophone-receivers that produce electrical output signals transmitted as input data, for example, to a computer 118 on a seismic control truck 120. Based on the input data, the computer 118 may generate a seismic data output, for example, a seismic two-way response time plot.

The seismic surface waves 115 travel more slowly than seismic body waves 114. Analysis of the time it takes seismic surface waves 115 to travel from source to sensor can provide information about near surface features.

A control center 122 can be operatively coupled to the seismic control truck 120 and other data acquisition and wellsite systems. The control center 122 may have computer facilities for receiving, storing, processing, and analyzing data from the seismic control truck 120 and other data acquisition and wellsite systems that provide additional information about the subsurface formation. For example, the control center 122 can receive data from a computer 119 associated with a well logging unit 121. For example, computer systems 124 in the control center 122 can be configured to analyze, model, control, optimize, or perform management tasks of field operations associated with development and production of resources such as oil and gas from the subsurface formation 100. Alternatively, the computer systems 124 can be located in a different location than the control center 122. Some computer systems are provided with functionality for manipulating and analyzing the data, such as performing seismic interpretation or borehole resistivity image log interpretation to identify geological surfaces in the subsurface formation or performing simulation, planning, and optimization of production operations of the wellsite systems.

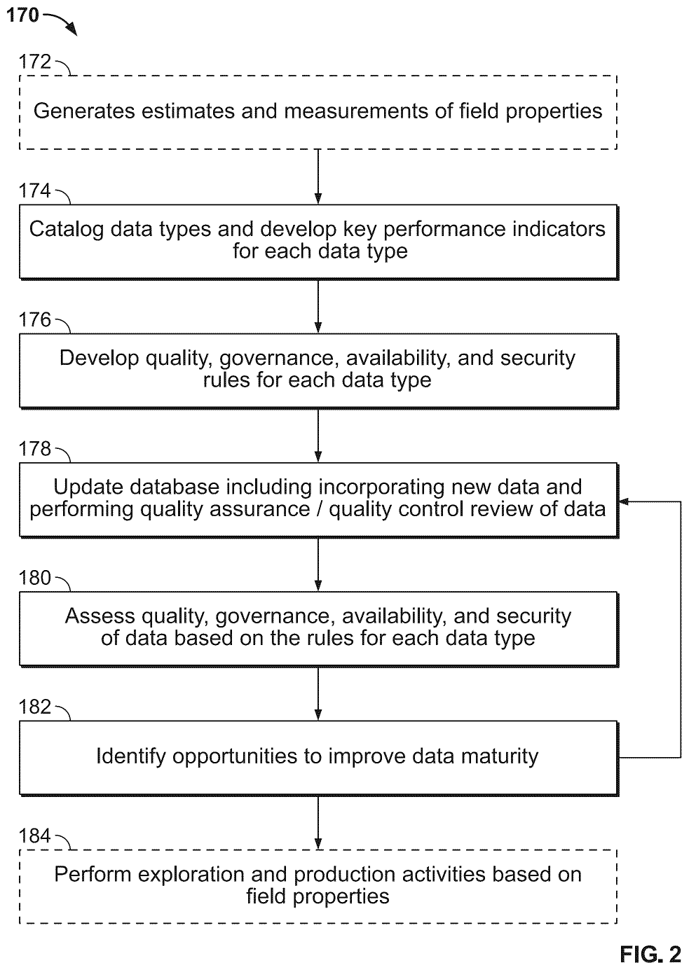

These computer systems or other computer systems not co-located with the field can be used to implement the systems and methods for supporting hydrocarbon exploration and production activities based on assessing the maturity of data types described with reference to FIGS. 2-4B. These systems and method are described with respect to data types associated with hydrocarbon exploration and production, however these systems and methods can be applied to other data types produced by an organization.

FIG. 2 is a flow diagram illustrating an approach to assessing the maturity of data. The approach is illustrated as a method 170 for supporting hydrocarbon exploration and production activities based on assessing the maturity of data types being used to develop and implement exploration and production strategies for a hydrocarbon field. The method can also be for assessing the maturity of other types of data.

The method 170 optionally includes generating estimates and measurements of field properties of a subsurface reservoir of the hydrocarbon field (step 172). In some implementations, the method 170 is performed on data (e.g., the estimates and measurements of field properties of a subsurface reservoir) that were previously generated without performing activities to generate new data.

Data types of the data (e.g., the estimates and measurements of field properties of a subsurface reservoir) are cataloged and key performance indicators are developed for each data type (step 174). For example, the data types can be based on data proponent, data users, data sources and data formats. The catalog helps to maintain and improve data integration and data consistency. For example, one organization may have separate departments/divisions in which multiple data types that are produced. An organization that produces data is the proponent of that data. The catalog is reviewed on a regular basis with the data proponent. Data types' formats are used to identify in which master repository (e.g., tabular repository, document repository, geospatial repository, seismic repository) that data should be stored in. A data type can have one or many formats (e.g. tabular, document, geospatial-vector, etc.). This catalog acts as the basis in which the following QGAS key performance indicators are measured.

In a prototype developed for supporting hydrocarbon exploration and production activities, the data types included includes more than 150 data types. These data types were grouped in the Exploration data taxonomy into five major categories of; wells, sub-surface, surface geology, geophysical and geospatial. The key data types include well, seismic, core, logs, tops, reports, reservoir, geospatial data. The data types established by building the business processes as part of the Enterprise Architecture (EA) then engaging the data producers (proponents) to identify and define each single data used as an input or output to that business processes.

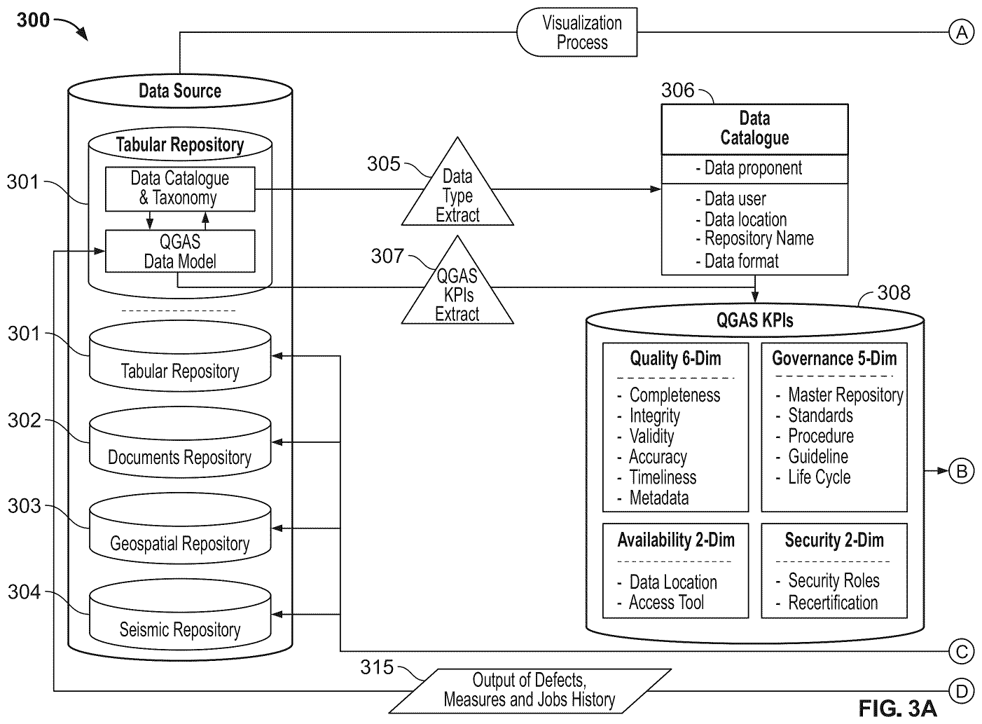

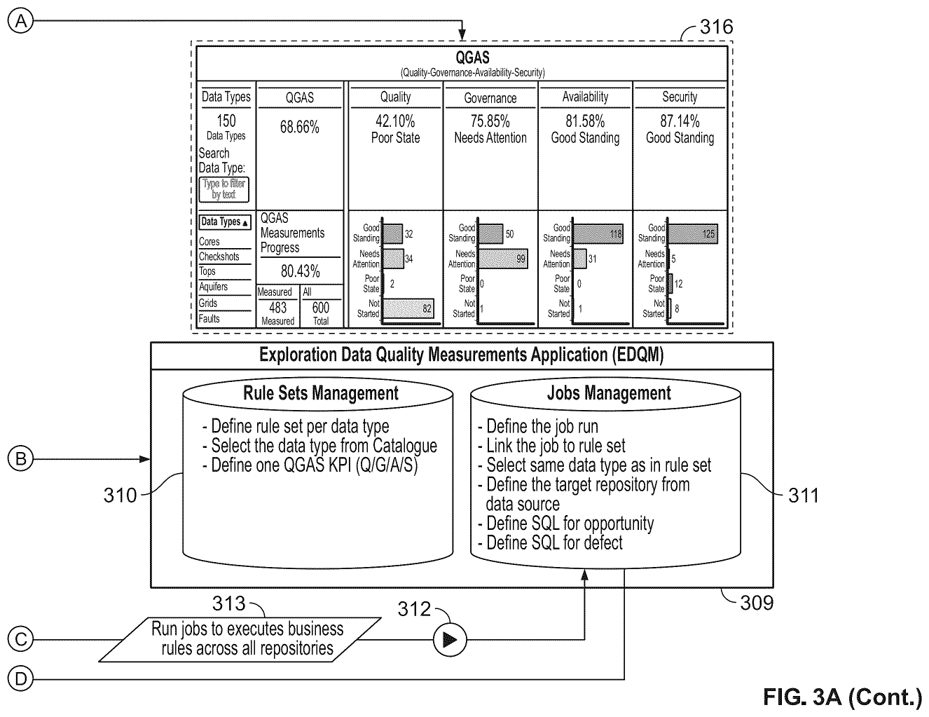

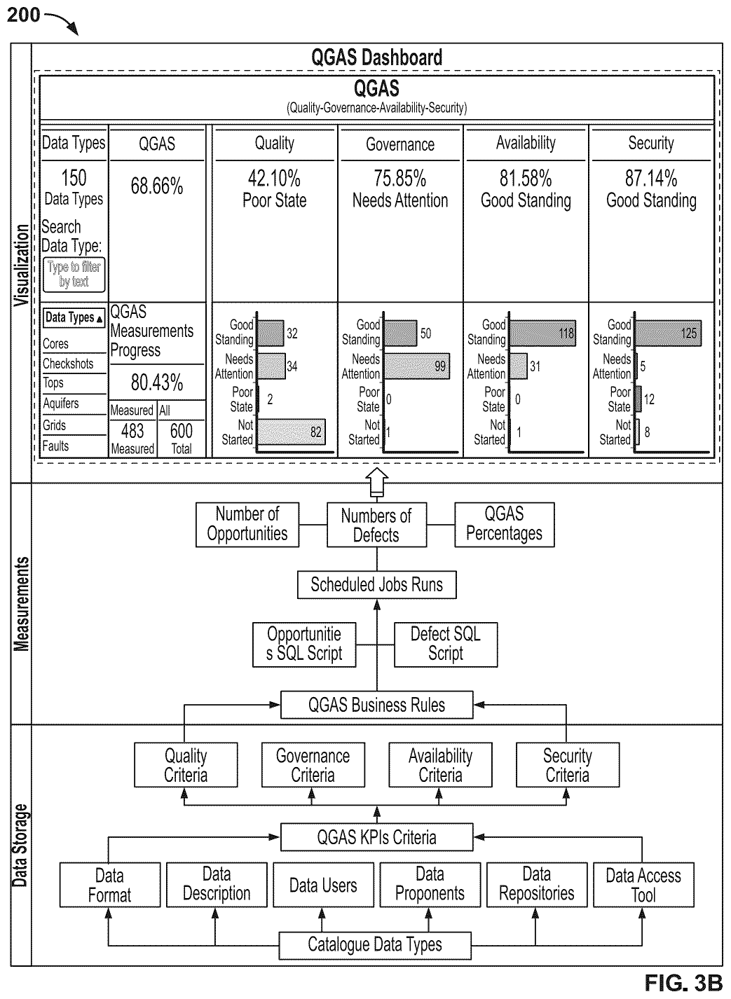

In the prototype, key performance indicators for quality, governance, availability and security were identified for each data type and stored in the corporate database (tabular repository) illustrated in FIG. 3B. The quality KPIs were converted to business rules and generated for each data type based on the data quality dimensions (completeness, integrity, validity, accuracy, timeliness, metadata). The business rules stored back in the tabular repository. For the governance, availability and security; the business rules equivalent to KPIs and generated one time to be used for all data types and stored in the same tabular repository. The measuring of the KPIs start by selecting the data type from Data Catalogue using Exploration Data Quality Measurements Application (EDQM). The generated business rules were defined in the rules sets section of EDQM for each data type using SQL. Then, the EDQM allows to create job run to check the master repositories against these rules, this will generate results of opportunities and defects which used by EDQM to calculate the percentages (number of defect divided by opportunity). The final results of defects, opportunity, measured scores and job runs will store again into the tabular repository to be used later by the dashboard.

Based on the key performance indicators, quality, governance, availability, and security rules are developed for each data type (step 176). The KPIs for quality starts by checking whether the data type is in the catalogue. If it doesn't exist, the default quality score is zero. Otherwise, SQL business rules is generated for each data types to check whether this data is complete, consistent, valid, accurate, and timely. A new SQL is created every time generating the quality business rules. The score measured as percentage of defects over the opportunities and the results ranged based on six-sigma categories, the final results stored in the tabular repository. Where in the cases of KPIs for governance, availability and security, the approach is slightly different. For these KPIs, the business rules equivalent to KPIs are generated using one SQL code for governance, one for availability and one security. The business rule for governance checking if the data types has a mater repository, standard, procedure, guideline and lifecycle, where the business rule for availability is checking whether the data type has located in the central repository and has application or tool to access this data. For the security the business rules inspect if the data has a security role and certified by the data proponent. All these business rules are used to measure scores for all data types in the catalogue using EDQM application and the final results stored back in the tabular repository. The stored measurements are reflected into QGAS dashboard.

Quality measurement methods can vary from one organization to another. For example, some organizations use six-sigma standards Whatever quality method is used, the score should be normalized to a 0 to 100% scale and fed into the model for a data type.

A database is updated, for example, by incorporating new data and performing quality assurance/quality control review of data (step 178).

The quality, governance, availability, and security of the data is assessed based on the rules for each data type (step 180). For example, the prototype was based on an excel sheet that includes five attributes. Quality, governance, availability, security estimated percentages were used to calculate the QGAS for each data type. For example, to estimate the security score for well core data, the prototype checks to see if the well core has database roles or not; it will get 70% out of 70% if it has a role according to the security KPIs, then will check if this role has certified or not and it will get 0% out of 30% if not certified; then the final score will be 70% out of 100%. The scores in the sheet were estimated for each data types with the same concepts for security, governance and availability. But for quality score, the percentage were populated from the data quality application (EDQM). The final percentages were used for testing the QGAS dashboard.

This assessment provides the basis for identifying opportunities to improve data maturity (step 182). For example, this assessment may identify geographic locations or data types where additional estimates and measurements of field properties of a subsurface reservoir can to be performed to improve data maturity. In another example, this assessment may identify when developing of additional tools to access data and/or performing additional quality assurance/quality control reviews can improve data maturity. Identifying why low QGAS scores are low provides an opportunity for engagement of the stakeholders to inspect and look for solutions as the part of monitoring group providing great opportunity. In another approach, a Data Ambassadors team with the main purpose to focus on reviewing the business rules with data owners then fixing the defects that leads to intensive assessment and inspection which eventually lead to improvement in data maturity.

The method 170 optionally includes performing exploration and production activities based on field properties (step 184). In some implementations, the exploration and production activities include, for example, drilling testing wells, performing seismic surveys, or implementing enhanced oil recovery technologies. For example, exploration activities can be performed to address gaps in data identified in the assessment of the quality, governance, availability, and security of the data based on the rules for each data type. In some implementations, the method 170 is performed on data (e.g., the estimates and measurements of field properties of a subsurface reservoir) that were previously generated without performing activities to generate new data.

FIGS. 3A and 3B are schematics illustrating the components of a system 300 implementing this method. A prototype system implementing this system was developed and implemented for supporting hydrocarbon exploration and production activities based on assessing the maturity of data types being used to develop and implement exploration and production strategies for a hydrocarbon field.

FIGS. 3A and 3B illustrate the concepts of implementation of QGAS measurements and the calculation of QGAS KPIs. The data sources for all Exploration data types exist in four repositories (tabular repository 301, document repository 302, geospatial repository 303, seismic repository 304). These data types were defined in the data catalogue and stored along with QGAS KPIs in the tabular repository 301.

For example, measurements can perform using an in-house quality application (EDQM) 309. The process is started by defining rules sets 310 for the data type. The data type is selected directly from the data catalogue 305 by extracting the required attributes 306. The other part of building the rules sets to identify which of KPIs are checked occurs between the data source and the data catalogue 307 and stored in the KPI database 308.

The second step in EDQM, is to build or paste the SQL code checking business rule for the data type selected in the rules set management database 310. The business rule editor has two important components: one for the SQL body and other for SQL WHERE statement which define the expected defects. The job also defined in this step to able automating the running of business rules using job frequency. The job run is executed in step 312 against one or some of the repositories 301, 302, 303 and 304 based on the data type location. The results of job execution 313 will go directly to EDQM 311 and a message it pop-up to confirm whether the job it runs successfully.

The last step in EDQM application 315 is to store back all the results of measurements into QGAS database tables in 301. These results include number of opportunities, defects, the calculated scores (defects divide by opportunities) %, jobs run numbers, SQL business rules body and attributes of the data type all of these will be stored back into the tabular repository.

The stored QGAS measurements in 301 can be visualized through a dashboard 316 (shown in more detail in FIG. 3B). All business rules for one data type are averaged to give QGAS for that data type and all data types belonging to one division are averaged to give the QGAS score for that division, and all divisions' QGAS scores are averaged to give QGAS score for the department, and all Exploration Department's QGAS scores were averaged to give the overall QGAS score and maturity level for Exploration data.

The system 300 populates all measurements in one data model and uses SQL queries to provide a data maturity measurement solution that measures complex business rules in quality, governance, availability and security.

In the prototype, the system 300 assesses data stored in one or more master repositories. Master repositories are centralized locations for storage of specific types of data. Storing data in master repositories can provide better ease of backup, quality assurance, and access than data stored at multiple discrete locations without central control and standards.

### Quality Key Performance Indicator

As previously mentioned, quality measurement methods can vary from one organization to another. Whatever quality method is used, the score should be normalized to a 0 to 100% scale and fed into the model for a data type.

In the prototype, if none of the formats of a data type are stored in a master repository, quality gets an automatic zero. Otherwise, quality is scored based on the quality measurements, which utilize six-sigma as a way for measurement for all master repositories. In this implementation, the key performance indicator is a Six-Sigma Quality Measurement. The key performance indicator looks at all the business rules defined for all master repositories in which a data type should be stored in based on its format(s). For each rule, the identified defects are divided over opportunities which results in the six-sigma quality score from 0 to 100%. The quality scores for each rule for a data type are averaged to obtain the overall quality score for that data type (e.g., see Eqn. 1).

$$Q for a data type = \frac{{\sum }_{i = 1}^{r} ( Quality \% for {B}_{r} )}{r} \qquad Eqn . 1$$

The prototype uses this score to define the quality state as noted shown in Table 1.

TABLE 1
Quality State

| From (%) | To (%) | Six-sigma level | State |
|---|---|---|---|
| 0 | | | Not Started |
| 1 | 68.9999 | 1σ | Poor State |
| 69.0000 | 99.3799 | 2σ and 3σ | Needs Attention |
| 99.3800 | 100 | 4σ to 6σ | Good Standing |

The quality process includes:
1. Identify all master repositories in which a data type should be stored in based on its format(s).
2. Analyze and capture data quality business rules in terms of completeness, integrity, conformity, timeliness, consistency, accuracy and meta-data. For example, to check the validity of formation tops that should be within the wellbore total depth, a quality business rule is generated with an English statement similar to: “The Maximum Top Pick Depth is Deeper than Wellbore Total Depth”. This simple English of business rule is converted to SQL statement and tested against the master repositories. This SQL then pasted into EDQM application 309 in FIG. 3A and run to check the data sources to see if there is any formation top depth deeper than the well total depth. Positive results are recorded as defects and save back into database 301. The job can be managed to run automatically through setting the job frequency either daily, weekly, bi-weekly or monthly. This automation is done for all business rules generated to measure the KPIs of QGAS.
3. Divide the resulted defects over opportunities for each business rule.
4. Average the resulted percentages to get the six-sigma quality score.
5. Reflect the score in the dashboard for tracking progress of Quality. For example, assume Data Type 1 has three formats; F₁, F₂ and F₃ that should be stored in three master repositories; R₁, R₂ and R₃ respectively. Example results of the six-sigma quality business rules are implemented on each one of the master repositories presented in Table 2.

If one data type has different formats, it will be stored in multiple repository. For example, well core has depth data (tabular repository), report data (document repository) and geospatial data (geospatial repository). In this case, each repository has different SQL for the same business rule. The final results will be based on averaging all percentage score measured in EDQM application.

TABLE 2
Example Quality Results

| | Master | Business | Count | Count | Defects/ | Quality % |
|---|---|---|---|---|---|---|
| Form. | Rep. | Rules | of | of | Opp. | (100 − |
| (Fₙ) | (Rₙ) | (Bᵣ) | Opp. | Defects | (DO) | DO) |
| F₁ | R₁ | B₁ | 100,000 | 5,000 | 5.00% | 95.00% |
| F₁ | R₁ | B₂ | 50,000 | 2,000 | 4.00% | 96.00% |
| F₂ | R₂ | B₃ | 80,000 | 2,000 | 2.50% | 97.50% |
| F₂ | R₂ | B₄ | 60,000 | 1,000 | 1.67% | 98.33% |
| F₃ | R₃ | B₅ | 70,000 | 3,000 | 4.29% | 95.71% |

Business rules in this example are assumed to have the same weight. However, some implementations give more weight to the more important business rules and less weight to the less important rules. Currently, no weighting was implemented in quality but all other QGAS has pre-weighting identified in the KPIs. For example, in governance; if the data types has a master repository it scores 60% where the rest of the four KPIs (standard, procedure & process, guideline and lifecycle) are weighted 10% for each. The same weighting approach is used in availability and security KPIs. Using Eqn. 1, Q for Data Type

$$1 = \frac{95.00 + 96.00 + 9750 + 98.33 + 95.71}{5} = 96.51 \% .$$

The quality score for Data Type 1 is 3σ, therefore, Data Type 1 is in a “Needs Attention” state.

Low scores can indicate many or significant defects that need to be fixed for that data types. Higher score can trigger adding additional business rules to increase the level of confident and to make data readiness for AI use.

### Governance Key Performance Indicator

In the prototype, if none of the formats of a data type are stored in a master repository, governance gets an automatic zero. Otherwise, governance is scored based on the key performance indicators in Table 3.

TABLE 3
Governance key performance indicators

| | Key performance | | Score |
|---|---|---|---|
| # | indicator | Description | (%) |
| 1 | Master Repository* | The data type has one or more | 60 |
| | | master repositories based on its | |
| | | format(s) | |
| 2 | Standard | The data type has documented | 10 |
| | | standards. The data type conforms | |
| | | to the standards. | |
| 3 | Procedure & Process | The data type has documented | 10 |
| | | procedures and/or processes. The | |
| | | data type conforms to them. | |
| 4 | Guideline | The data type has documented | 10 |
| | | guidelines. The data type conforms | |
| | | to them. | |
| 5 | Life Cycle | The data has a managed life cycle | 10 |

The “Master Repository” key performance indicator is dependent on the number of master repositories a data type should be stored in and will be averaged towards the 60% score assigned to that key performance indicator. For example, if a data type should be stored in 2 master repositories; R₁ and R₂ based on its 2 formats; F₁ and F₂, then both repositories will split the score assignment and will get 30% each. If F₁ is stored in R₁ but F₂ is not stored in R₂, then F₁/R₁ will get 30% and F₂/R₂ will get 0%. These scores for each key performance indicator are summed to calculate the governance score for a particular data type (e.g., see Eqn. 2).

$$G for a data type = 60 ( \frac{{\sum }_{i = 1}^{n} {R}_{n}}{n} ) + 10 ( 1 if ST = Yes , else 0 ) + 10 ( 1 if PP = Yes , else 0 ) + 10 ( 1 if GI = Yes , else 0 ) + 10 ( 1 if LC = Yes , else 0 ) \qquad Eqn . 2$$

$$where {R}_{n} = \{ \begin{matrix}1 , & RL = Yes \\ 0 , & RL = No\end{matrix}$$

The prototype uses this score to define the quality state as noted shown in Table 4.

TABLE 4
Governance State

| | From (%) | To (%) | State |
|---|---|---|---|
| 0 | | | Not Started |
| | 1 | 29 | Poor State |
| | 30 | 89 | Needs Attention |
| | 90 | 100 | Good Standing |

The governance process includes:
1. Identify all master repositories in which a data type should be stored in based on its format(s).
2. Identify the exact location in which the data type is stored. A location can vary depending on the master repository type and setup. The objective is to be able to reference the data type in a specific master repository.
3. Identify all documentations for standards, procedures & processes and guidelines related to the data type.
4. Assign the scores according to the key performance indicators criteria in Table 3.
5. Reflect the score in the dashboard for tracking progress of governance.

For example, assume Data Type 1 has three formats; F₁, F₂ and F₃ that should be stored in three master repositories; R₁, R₂ and R₃ respectively. Example results of the governance analysis are presented in Table 5.

TABLE 5
Example Governance Results

| | | In Master | | Has | Has | Has |
|---|---|---|---|---|---|---|
| | Master | Repo. and | Has | Procedure/ | Guide- | Life |
| Formats | Repo. | Location is | Standard | Process | line | Cycle |
| (Fₙ) | (Rₙ) | known (RL) | (ST) | (PP) | (GI) | (LC) |
| F₁ | R₁ | Yes | Yes | No | Yes | No |
| F₂ | R₂ | Yes | Yes | No | Yes | No |
| F₃ | R₃ | No | Yes | No | Yes | No |

Using Eqn. 1,

$$G for Data Type 1 = 60 ( \frac{1 + 1 + 0}{3} ) + 10 ( 1 ) + 10 ( 0 ) + 10 ( 1 ) + 10 ( 0 ) = 60 \% .$$

Therefore, Data Type 1 is in a “Needs Attention” state for governance.

Low governance results indicate that the data governance is missing the higher KPIs weighting associated with storing data in known master repository. In this case, an immediate plan should be implemented to capture that data type into a master repository.

### Availability Key Performance Indicator

In contrast to quality and governance, availability can be measured even if none of the formats of a data type are stored in a master repository, this is because data types can still be available to the business even without measuring and monitoring. Availability is scored based on whether the data is stored in a central location and whether the data type has an associated access tool (e.g., see the key performance indicators in Table 6).

TABLE 6
Availability key performance indicators

| | Key performance | | Score |
|---|---|---|---|
| # | indicator | Description | (%) |
| 1 | Central Location | The data type has a central | 30 |
| | | location that is recognized by the | |
| | | data proponents. This could be a | |
| | | master repository, application, | |
| | | shared folder, etc. | |
| 2 | Access Tool | The data type has the right tool to | 70 |
| | | make it available (access/manage) | |

In the prototype, both key performance indicators are dependent on the number of formats a data type comes in and will be averaged towards the 30% and 70% scores assigned to these key performance indicators. For example, if a data type comes in 2 formats; F₁ and F₂ then both formats will split the score assignments and will get 15% each for the first key performance indicator and 35% each for the second key performance indicator (e.g., see Eqn. 3).

$$A for a data type = 30 ( \frac{{\sum }_{i = 1}^{n} {F}_{n} CL}{n} ) + 70 ( \frac{{\sum }_{i = 1}^{n} {F}_{n} T}{n} ) where {F}_{n} CL = \{ \begin{matrix}1 , & CL = Yes \\ 0 , & CL = No\end{matrix} \qquad Eqn . 3$$

$$and {F}_{n} T = \{ \begin{matrix}1 , & T = Yes \\ 0 , & T = No\end{matrix}$$

The “Access Tool” key performance indicator will get an automatic zero if the corresponding format does not have a “Central Location”.

The prototype uses this score to define the availability state as noted shown in Table 6.

TABLE 6
Availability State

| | From (%) | To (%) | State |
|---|---|---|---|
| 0 | | | Not Started |
| | 1 | 29 | Poor State |
| | 30 | 69 | Needs Attention |
| | 70 | 100 | Good Standing |

The availability process includes:
1. Identify all formats a data type comes in.
2. Identify all central locations (master or non-master) in which the data type is stored and are recognized by the data proponents.
3. Identify access tools that can enable data users to access the different formats of a data type.
4. Assign the scores according to the key performance indicators criteria above.
5. Reflect the score in the dashboard for tracking progress of Availability.

For example, assume Data Type 1 has three formats; F₁, F₂ and F₃ that should have one or more central locations that are recognized by the data proponent. They should also be accessible to users by data access tools. Example results are presented in Table 7.

TABLE 7
Example Availability Results

| | Has a Central | Can be accessed |
|---|---|---|
| Formats (Fₙ) | Location (CL) | from a tool (T) |
| F₁ | Yes | Yes |
| F₂ | Yes | No |
| F₃ | No | No |

Examples of central location include sharefolders that including important organizational data, but not available or accessible to all authorized users. This also including some applications that have a closed box of storing the output data which not directly captured into the master data repositories. A central location is a bigger data container that including all master repositories, sharefolders, applications databases or any known data storage.

Using Eqn. 3,

$$A for Data Type 1 = 30 ( \frac{1 + 1 + 0}{3} ) + 70 ( \frac{1 + 0 + 0}{3} ) = 43 \% .$$

Therefore, Data Type 1 is in a “Needs Attention” state for availability.

Availability scores trigger some cases where the data exist in a central location but not all users able accessing this data, either have no idea about this location or they do not have an appropriate applications or tool to access this data type.

### Security Key Performance Indicator

Like quality and governance, security gets an automatic zero if none of the formats of a data type are stored in a master repository, Otherwise, security is scored based on the key performance indicators in Table 8.

TABLE 8
Security key performance indicators

| | Key performance | | Score |
|---|---|---|---|
| # | indicator | Description | (%) |
| 1 | Security Roles | The data type has all needed | 70 |
| | | security roles in the master | |
| | | repositories in which a data type | |
| | | should be stored in based on its | |
| | | format(s) | |
| 2 | Roles Certification* | The security roles have a valid | 30 |
| | | certification | |

The “Security Roles” key performance indicator is dependent on the roles available in the different master repositories a data type should be stored in and will be averaged towards the 70% score assigned to that key performance indicator. For example, if a data type should be stored in 2 master repositories; R₁ and R₂ based on its 2 formats; F₁ and F₂, then both repositories will split the score assignment and will get 35% each. If F₁ is stored in R₁ and has valid security roles but F₂ is stored in R₂ without valid security roles, then F₁/R₁ will get 35% and F₂/R₂ will get 0%. These scores for each key performance indicator are summed to calculate the governance score for a particular data type (e.g., see Eqn. 4).

$$S for a data type = 70 ( \frac{{\sum }_{i = 1}^{n} {R}_{n}}{n} ) + 30 ( 1 if CT = Yes , else 0 ) \qquad Eqn . 4$$

$$where {R}_{n} = \{ \begin{matrix}1 , & SR = Yes \\ 0 , & SR = No\end{matrix}$$

Security roles validation and certifications policies and methods can vary from one organization to another. As long as there are clear definitions, the key performance indicator will deem useful for measuring the maturity of the security. For example, a department role can be created to bundle all privileges access to department data in master repositories. When new employees join any associated department, the role is automatically assigned to these employees. All roles are typically sent to the data owner periodically for review and certification of who can access their data.

The prototype uses this score to define the quality state as noted shown in Table 9.

TABLE 9
Security State

| | From (%) | To (%) | State |
|---|---|---|---|
| 0 | | | Not Started |
| | 1 | 69 | Poor State |
| | 70 | 79 | Needs Attention |
| | 80 | 100 | Good Standing |

The range for quality is based on six-sigma protocols, where the ranges for governance, availability and security were defined based on the weighting of KPIs. If the higher KPIs weighting exist in the data types, this data type would be in a good condition. If the medium KPIs weighting exits, this indicates more attention is required, otherwise, it would be in a poor state.

### The Security Process Includes:

1. Identify all master repositories in which a data type should be stored in based on its format(s).

2. Identify the exact location in which the data type is stored. A location can vary depending on the master repository type and setup. The objective is to be able to reference your data type in a specific master repository.

3. Identify security roles on these locations and check if there are any violation.

4. Check if these security roles have a valid certification.

5. Assign the scores according to the key performance indicators criteria in Table 9.

6. Reflect the score in the dashboard for tracking progress of security.

For example, assume Data Type 1 has three formats; F₁, F₂ and F₃ that should be stored in three master repositories; R₁, R₂ and R₃ respectively. For each master repository, security roles have to be valid and certified. Example results of the governance analysis are presented in Table 10.

TABLE 10
Example Security Results

| | | Has Valid Security | Roles are |
|---|---|---|---|
| Formats (Fₙ) | Master Repo. (Rₙ) | Roles (SR) | Certified (CT) |
| F₁ | R₁ | Yes | No |
| F₂ | R₂ | Yes | No |
| F₃ | R₃ | No | No |

Using Eqn. 4,

$$S for Data Type 1 = 70 ( \frac{1 + 1 + 0}{3} ) + 30 ( 0 ) = 47 \% .$$

Therefore, Data Type 1 is in a “Poor” state for security.

Security scores can indicate issues such as when employee re-assigned to department out of the exploration organization without their access privileges being revoked thus flagging individuals accessing exploration data from outside the exploration organization.

### Overall Score

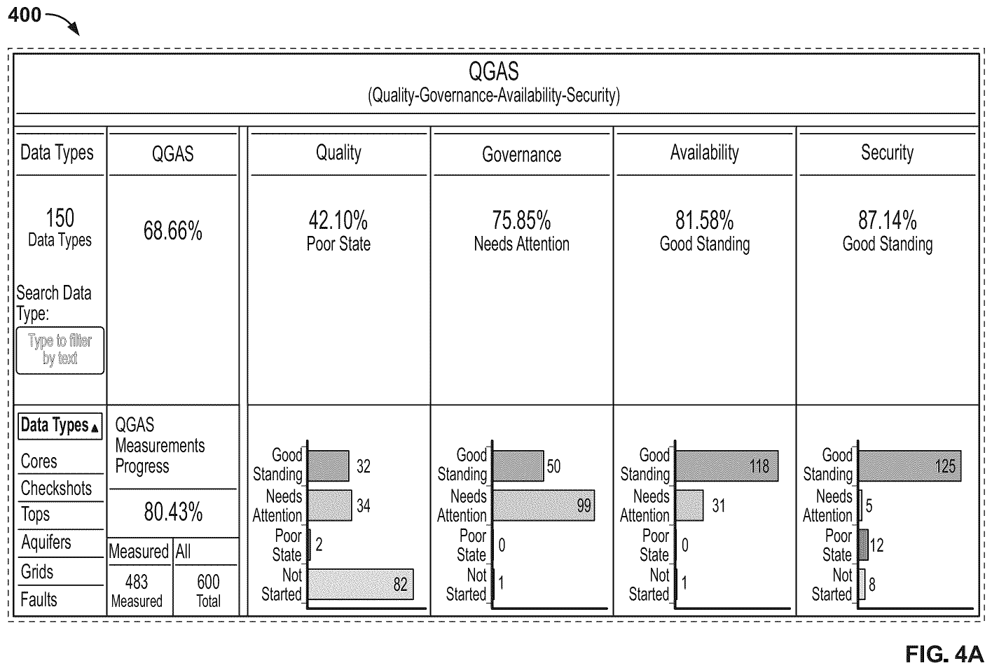

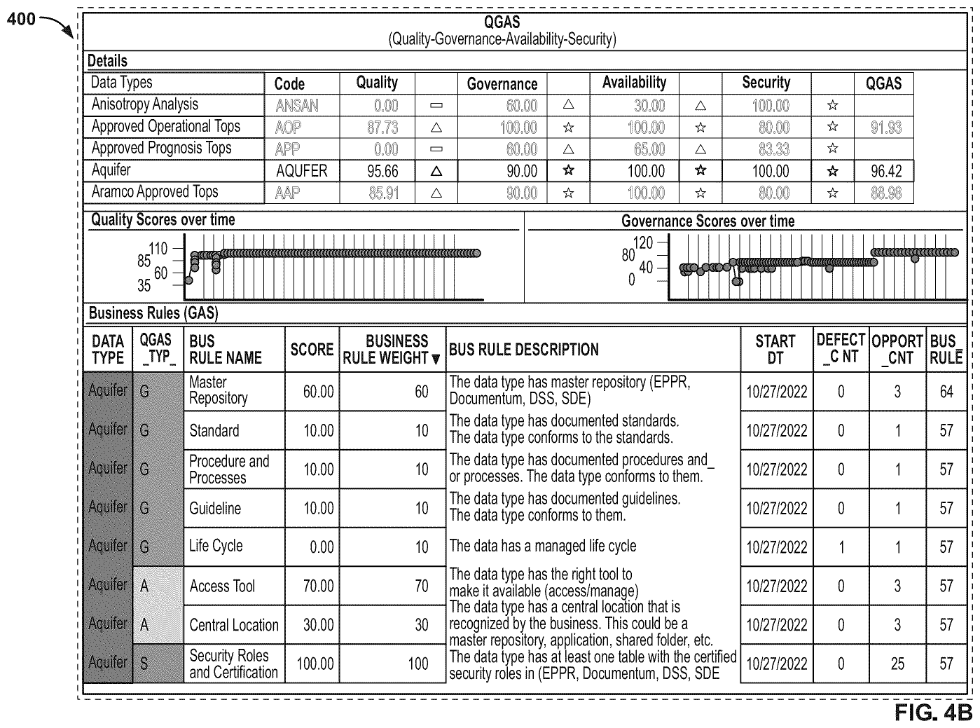

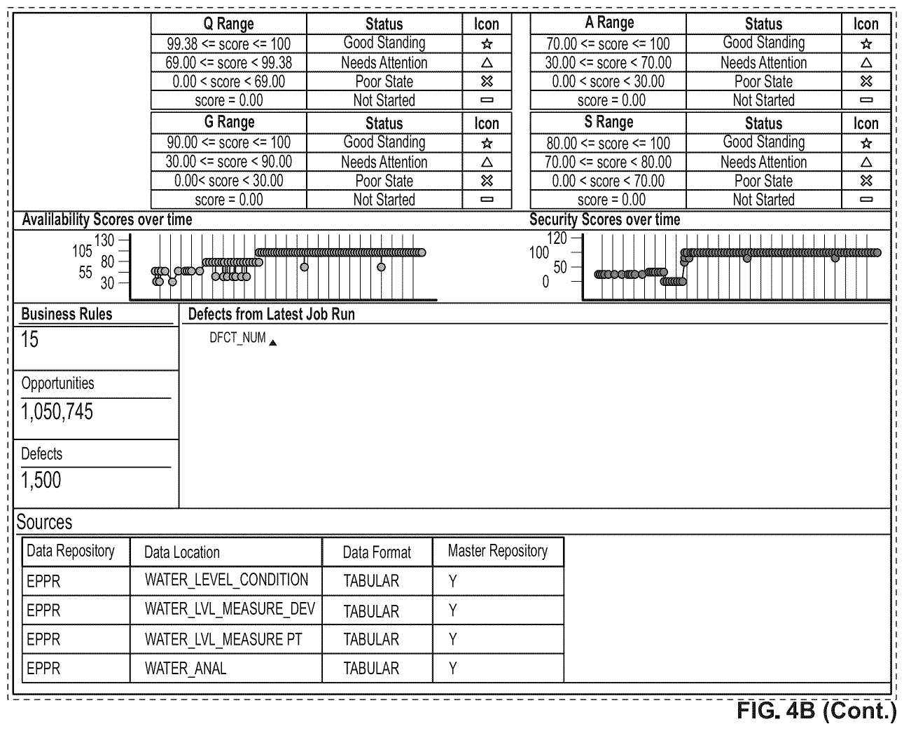

The overall data maturity score is calculated to measure how good and mature the data is. Each component of score measurement is scored separately from 0-100% based on specific key performance indicators designed to simplify the process and clearly indicate areas for possible improvements. All four component scores are averaged together into one overall score for a single data type. This score provides a clear view on the strengths and the areas of improvements for each data type in an organization via a dashboard. The dashboard for the prototype is described with respect to FIGS. 4A and 4B.

Although an overall score is measuring the maturity of a single data type, it can cascade to give a score for the data proponent at any organizational level. An example is presented in Table 11.

TABLE 11
Example overall data scores

| | | Q | G | A | S | Overall |
|---|---|---|---|---|---|---|
| Organization | Data Type | (%) | (%) | (%) | (%) | (%) |
| Org1 | DT 1 | 90 | 60 | 30 | 70 | 63 |
| | DT 2 | 70 | 90 | 100 | 70 | 83 |
| | DT 3 | 0 | 0 | 100 | 0 | 25 |
| | Average | 53 | 50 | 77 | 47 | 57 |
| Org2 | DT 2* | 70 | 90 | 100 | 70 | 83 |
| | DT 4 | 75 | 100 | 30 | 70 | 69 |
| | Average | 73 | 95 | 65 | 70 | 76 |

*Multiple organizations can be producing the same data type. A data type is scored regardless of the different data proponents.

In general, a data type can have one or more formats and can be stored in one or more master repositories. It is up to an organization to decide what formats needs to be measured and what data repositories are considered master repositories. The four states are defined to give an indication of the data maturity in each of the components (e.g., quality, governance, availability, and security in the prototype). These states are heavily dependent on are deemed more important in the key performance indicators. For example, in the prototype governance key performance indicators, the availability of a master repository will give the data type a 70% score and will move its G state from “Poor State” to “Needs Attention”. However, in the quality key performance indicators any six-sigma score between 1% and 93.29% is considered to be in “Poor State”. A zero score for any of the QGAS components indicate a “Not Started” state which is very important as data types usually have some kind of fulfillments to the key performance indicators but not yet measured.

FIGS. 4A and 4B are screen shots of the graphic user interface 200 of a prototype system implementing the approach of FIG. 2.

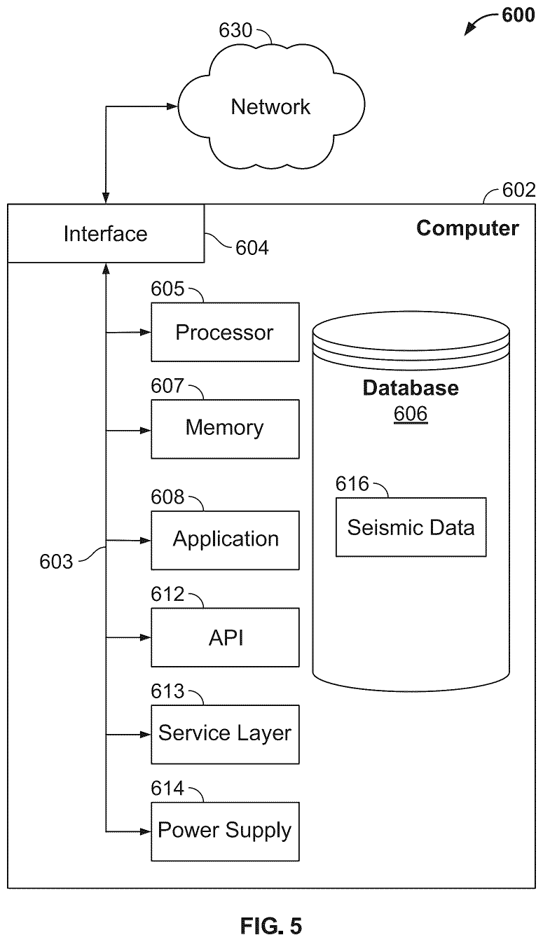

FIG. 5 is a block diagram of an example computer system 600 used to provide computational functionalities associated with described algorithms, methods, functions, processes, flows, and procedures described in the present disclosure, according to some implementations of the present disclosure. The illustrated computer 602 is intended to encompass any computing device such as a server, a desktop computer, a laptop/notebook computer, a wireless data port, a smart phone, a personal data assistant (PDA), a tablet computing device, or one or more processors within these devices, including physical instances, virtual instances, or both. The computer 602 can include input devices such as keypads, keyboards, and touch screens that can accept user information. Also, the computer 602 can include output devices that can convey information associated with the operation of the computer 602. The information can include digital data, visual data, audio information, or a combination of information. The information can be presented in a graphical user interface (UI) (or GUI).

The computer 602 can serve in a role as a client, a network component, a server, a database, a persistency, or components of a computer system for performing the subject matter described in the present disclosure. The illustrated computer 602 is communicably coupled with a network 630. In some implementations, one or more components of the computer 602 can be configured to operate within different environments, including cloud-computing-based environments, local environments, global environments, and combinations of environments.

At a high level, the computer 602 is an electronic computing device operable to receive, transmit, process, store, and manage data and information associated with the described subject matter. According to some implementations, the computer 602 can also include, or be communicably coupled with, an application server, an email server, a web server, a caching server, a streaming data server, or a combination of servers.

The computer 602 can receive requests over network 630 from a client application (for example, executing on another computer 602). The computer 602 can respond to the received requests by processing the received requests using software applications. Requests can also be sent to the computer 602 from internal users (for example, from a command console), external (or third) parties, automated applications, entities, individuals, systems, and computers.

Each of the components of the computer 602 can communicate using a system bus 603. In some implementations, any or all of the components of the computer 602, including hardware or software components, can interface with each other or the interface 604 (or a combination of both), over the system bus 603. Interfaces can use an application programming interface (API) 612, a service layer 613, or a combination of the API 612 and service layer 613. The API 612 can include specifications for routines, data structures, and object classes. The API 612 can be either computer-language independent or dependent. The API 612 can refer to a complete interface, a single function, or a set of APIs.

The service layer 613 can provide software services to the computer 602 and other components (whether illustrated or not) that are communicably coupled to the computer 602. The functionality of the computer 602 can be accessible for all service consumers using this service layer. Software services, such as those provided by the service layer 613, can provide reusable, defined functionalities through a defined interface. For example, the interface can be software written in JAVA, C++, or a language providing data in extensible markup language (XML) format. While illustrated as an integrated component of the computer 602, in alternative implementations, the API 612 or the service layer 613 can be stand-alone components in relation to other components of the computer 602 and other components communicably coupled to the computer 602. Moreover, any or all parts of the API 612 or the service layer 613 can be implemented as child or sub-modules of another software module, enterprise application, or hardware module without departing from the scope of the present disclosure.

The computer 602 includes an interface 604. Although illustrated as a single interface 604 in FIG. 5, two or more interfaces 604 can be used according to particular needs, desires, or particular implementations of the computer 602 and the described functionality. The interface 604 can be used by the computer 602 for communicating with other systems that are connected to the network 630 (whether illustrated or not) in a distributed environment. Generally, the interface 604 can include, or be implemented using, logic encoded in software or hardware (or a combination of software and hardware) operable to communicate with the network 630. More specifically, the interface 604 can include software supporting one or more communication protocols associated with communications. As such, the network 630 or the hardware of the interface can be operable to communicate physical signals within and outside of the illustrated computer 602.

The computer 602 includes a processor 605. Although illustrated as a single processor 605 in FIG. 5, two or more processors 605 can be used according to particular needs, desires, or particular implementations of the computer 602 and the described functionality. Generally, the processor 605 can execute instructions and can manipulate data to perform the operations of the computer 602, including operations using algorithms, methods, functions, processes, flows, and procedures as described in the present disclosure.

The computer 602 also includes a database 606 that can hold data (for example, seismic data 616) for the computer 602 and other components connected to the network 630 (whether illustrated or not). For example, database 606 can be an in-memory, conventional, or a database storing data consistent with the present disclosure. In some implementations, database 606 can be a combination of two or more different database types (for example, hybrid in-memory and conventional databases) according to particular needs, desires, or particular implementations of the computer 602 and the described functionality. Although illustrated as a single database 606 in FIG. 5, two or more databases (of the same, different, or combination of types) can be used according to particular needs, desires, or particular implementations of the computer 602 and the described functionality. While database 606 is illustrated as an internal component of the computer 602, in alternative implementations, database 606 can be external to the computer 602.

The computer 602 also includes a memory 607 that can hold data for the computer 602 or a combination of components connected to the network 630 (whether illustrated or not). Memory 607 can store any data consistent with the present disclosure. In some implementations, memory 607 can be a combination of two or more different types of memory (for example, a combination of semiconductor and magnetic storage) according to particular needs, desires, or particular implementations of the computer 602 and the described functionality. Although illustrated as a single memory 607 in FIG. 5, two or more memories 607 (of the same, different, or combination of types) can be used according to particular needs, desires, or particular implementations of the computer 602 and the described functionality. While memory 607 is illustrated as an internal component of the computer 602, in alternative implementations, memory 607 can be external to the computer 602.

The application 608 can be an algorithmic software engine providing functionality according to particular needs, desires, or particular implementations of the computer 602 and the described functionality. For example, application 608 can serve as one or more components, modules, or applications. Further, although illustrated as a single application 608, the application 608 can be implemented as multiple applications 608 on the computer 602. In addition, although illustrated as internal to the computer 602, in alternative implementations, the application 608 can be external to the computer 602.

The computer 602 can also include a power supply 614. The power supply 614 can include a rechargeable or non-rechargeable battery that can be configured to be either user- or non-user-replaceable. In some implementations, the power supply 614 can include power-conversion and management circuits, including recharging, standby, and power management functionalities. In some implementations, the power-supply 614 can include a power plug to allow the computer 602 to be plugged into a wall socket or a power source to, for example, power the computer 602 or recharge a rechargeable battery.

There can be any number of computers 602 associated with, or external to, a computer system containing computer 602, with each computer 602 communicating over network 630. Further, the terms “client,” “user,” and other appropriate terminology can be used interchangeably, as appropriate, without departing from the scope of the present disclosure. Moreover, the present disclosure contemplates that many users can use one computer 602 and one user can use multiple computers 602.

Implementations of the subject matter and the functional operations described in this specification can be implemented in digital electronic circuitry, in tangibly embodied computer software or firmware, in computer hardware, including the structures disclosed in this specification and their structural equivalents, or in combinations of one or more of them. Software implementations of the described subject matter can be implemented as one or more computer programs. Each computer program can include one or more modules of computer program instructions encoded on a tangible, non transitory, computer-readable computer-storage medium for execution by, or to control the operation of, data processing apparatus. Alternatively, or additionally, the program instructions can be encoded in/on an artificially generated propagated signal. The example, the signal can be a machine-generated electrical, optical, or electromagnetic signal that is generated to encode information for transmission to suitable receiver apparatus for execution by a data processing apparatus. The computer-storage medium can be a machine-readable storage device, a machine-readable storage substrate, a random or serial access memory device, or a combination of computer-storage mediums.

The terms “data processing apparatus,” “computer,” and “electronic computer device” (or equivalent as understood by one of ordinary skill in the art) refer to data processing hardware. For example, a data processing apparatus can encompass all kinds of apparatus, devices, and machines for processing data, including by way of example, a programmable processor, a computer, or multiple processors or computers. The apparatus can also include special purpose logic circuitry including, for example, a central processing unit (CPU), a field programmable gate array (FPGA), or an application specific integrated circuit (ASIC). In some implementations, the data processing apparatus or special purpose logic circuitry (or a combination of the data processing apparatus or special purpose logic circuitry) can be hardware- or software-based (or a combination of both hardware- and software-based). The apparatus can optionally include code that creates an execution environment for computer programs, for example, code that constitutes processor firmware, a protocol stack, a database management system, an operating system, or a combination of execution environments. The present disclosure contemplates the use of data processing apparatuses with or without conventional operating systems, for example, LINUX, UNIX, WINDOWS, MAC OS, ANDROID, or IOS.

A computer program, which can also be referred to or described as a program, software, a software application, a module, a software module, a script, or code, can be written in any form of programming language. Programming languages can include, for example, compiled languages, interpreted languages, declarative languages, or procedural languages. Programs can be deployed in any form, including as stand-alone programs, modules, components, subroutines, or units for use in a computing environment. A computer program can, but need not, correspond to a file in a file system. A program can be stored in a portion of a file that holds other programs or data, for example, one or more scripts stored in a markup language document, in a single file dedicated to the program in question, or in multiple coordinated files storing one or more modules, sub programs, or portions of code. A computer program can be deployed for execution on one computer or on multiple computers that are located, for example, at one site or distributed across multiple sites that are interconnected by a communication network. While portions of the programs illustrated in the various figures may be shown as individual modules that implement the various features and functionality through various objects, methods, or processes, the programs can instead include a number of sub-modules, third-party services, components, and libraries. Conversely, the features and functionality of various components can be combined into single components as appropriate. Thresholds used to make computational determinations can be statically, dynamically, or both statically and dynamically determined.

The methods, processes, or logic flows described in this specification can be performed by one or more programmable computers executing one or more computer programs to perform functions by operating on input data and generating output. The methods, processes, or logic flows can also be performed by, and apparatus can also be implemented as, special purpose logic circuitry, for example, a CPU, an FPGA, or an ASIC.

Computers suitable for the execution of a computer program can be based on one or more of general and special purpose microprocessors and other kinds of CPUs. The elements of a computer are a CPU for performing or executing instructions and one or more memory devices for storing instructions and data. Generally, a CPU can receive instructions and data from (and write data to) a memory. A computer can also include, or be operatively coupled to, one or more mass storage devices for storing data. In some implementations, a computer can receive data from, and transfer data to, the mass storage devices including, for example, magnetic, magneto optical disks, or optical disks. Moreover, a computer can be embedded in another device, for example, a mobile telephone, a personal digital assistant (PDA), a mobile audio or video player, a game console, a global positioning system (GPS) receiver, or a portable storage device such as a universal serial bus (USB) flash drive.

Computer readable media (transitory or non-transitory, as appropriate) suitable for storing computer program instructions and data can include all forms of permanent/non-permanent and volatile/non-volatile memory, media, and memory devices. Computer readable media can include, for example, semiconductor memory devices such as random access memory (RAM), read only memory (ROM), phase change memory (PRAM), static random access memory (SRAM), dynamic random access memory (DRAM), erasable programmable read-only memory (EPROM), electrically erasable programmable read-only memory (EEPROM), and flash memory devices. Computer readable media can also include, for example, magnetic devices such as tape, cartridges, cassettes, and internal/removable disks. Computer readable media can also include magneto optical disks and optical memory devices and technologies including, for example, digital video disc (DVD), CD ROM, DVD+/−R, DVD-RAM, DVD-ROM, HD-DVD, and BLURAY. The memory can store various objects or data, including caches, classes, frameworks, applications, modules, backup data, jobs, web pages, web page templates, data structures, database tables, repositories, and dynamic information. Types of objects and data stored in memory can include parameters, variables, algorithms, instructions, rules, constraints, and references. Additionally, the memory can include logs, policies, security or access data, and reporting files. The processor and the memory can be supplemented by, or incorporated in, special purpose logic circuitry.

Implementations of the subject matter described in the present disclosure can be implemented on a computer having a display device for providing interaction with a user, including displaying information to (and receiving input from) the user. Types of display devices can include, for example, a cathode ray tube (CRT), a liquid crystal display (LCD), a light-emitting diode (LED), and a plasma monitor. Display devices can include a keyboard and pointing devices including, for example, a mouse, a trackball, or a trackpad. User input can also be provided to the computer through the use of a touchscreen, such as a tablet computer surface with pressure sensitivity or a multi-touch screen using capacitive or electric sensing. Other kinds of devices can be used to provide for interaction with a user, including to receive user feedback including, for example, sensory feedback including visual feedback, auditory feedback, or tactile feedback. Input from the user can be received in the form of acoustic, speech, or tactile input. In addition, a computer can interact with a user by sending documents to, and receiving documents from, a device that is used by the user. For example, the computer can send web pages to a web browser on a user's client device in response to requests received from the web browser.

The term “graphical user interface,” or “GUI,” can be used in the singular or the plural to describe one or more graphical user interfaces and each of the displays of a particular graphical user interface. Therefore, a GUI can represent any graphical user interface, including, but not limited to, a web browser, a touch screen, or a command line interface (CLI) that processes information and efficiently presents the information results to the user. In general, a GUI can include a plurality of user interface (UI) elements, some or all associated with a web browser, such as interactive fields, pull-down lists, and buttons. These and other UI elements can be related to or represent the functions of the web browser.

Implementations of the subject matter described in this specification can be implemented in a computing system that includes a back end component, for example, as a data server, or that includes a middleware component, for example, an application server. Moreover, the computing system can include a front-end component, for example, a client computer having one or both of a graphical user interface or a Web browser through which a user can interact with the computer. The components of the system can be interconnected by any form or medium of wireline or wireless digital data communication (or a combination of data communication) in a communication network. Examples of communication networks include a local area network (LAN), a radio access network (RAN), a metropolitan area network (MAN), a wide area network (WAN), Worldwide Interoperability for Microwave Access (WIMAX), a wireless local area network (WLAN) (for example, using 802.11 a/b/g/n or 802.20 or a combination of protocols), all or a portion of the Internet, or any other communication system or systems at one or more locations (or a combination of communication networks). The network can communicate with, for example, Internet Protocol (IP) packets, frame relay frames, asynchronous transfer mode (ATM) cells, voice, video, data, or a combination of communication types between network addresses.

The computing system can include clients and servers. A client and server can generally be remote from each other and can typically interact through a communication network. The relationship of client and server can arise by virtue of computer programs running on the respective computers and having a client-server relationship.

Cluster file systems can be any file system type accessible from multiple servers for read and update. Locking or consistency tracking may not be necessary since the locking of exchange file system can be done at application layer. Furthermore, Unicode data files can be different from non-Unicode data files.

While this specification contains many specific implementation details, these should not be construed as limitations on the scope of what may be claimed, but rather as descriptions of features that may be specific to particular implementations. Certain features that are described in this specification in the context of separate implementations can also be implemented, in combination, in a single implementation. Conversely, various features that are described in the context of a single implementation can also be implemented in multiple implementations, separately, or in any suitable sub-combination. Moreover, although previously described features may be described as acting in certain combinations and even initially claimed as such, one or more features from a claimed combination can, in some cases, be excised from the combination, and the claimed combination may be directed to a sub-combination or variation of a sub-combination.

Particular implementations of the subject matter have been described. Other implementations, alterations, and permutations of the described implementations are within the scope of the following claims as will be apparent to those skilled in the art. While operations are depicted in the drawings or claims in a particular order, this should not be understood as requiring that such operations be performed in the particular order shown or in sequential order, or that all illustrated operations be performed (some operations may be considered optional), to achieve desirable results. In certain circumstances, multitasking or parallel processing (or a combination of multitasking and parallel processing) may be advantageous and performed as deemed appropriate.

Moreover, the separation or integration of various system modules and components in the previously described implementations should not be understood as requiring such separation or integration in all implementations, and it should be understood that the described program components and systems can generally be integrated together in a single software product or packaged into multiple software products.

Accordingly, the previously described example implementations do not define or constrain the present disclosure. Other changes, substitutions, and alterations are also possible without departing from the spirit and scope of the present disclosure.

Furthermore, any claimed implementation is considered to be applicable to at least a computer-implemented method; a non-transitory, computer-readable medium storing computer-readable instructions to perform the computer-implemented method; and a computer system comprising a computer memory interoperably coupled with a hardware processor configured to perform the computer-implemented method or the instructions stored on the non-transitory, computer-readable medium.

A number of embodiments of the systems and methods have been described. Nevertheless, it will be understood that various modifications may be made without departing from the spirit and scope of the disclosure. Accordingly, other embodiments are within the scope of the following claims.

What is claimed is:

1. A method for hydrocarbon exploration of a subsurface reservoir based on assessing the maturity of data on the subsurface reservoir, the method comprising:
generating estimates and measurements of properties of the subsurface reservoir;
cataloging data types of the estimates and measurements of properties of the subsurface reservoir;
developing key performance indicators for each data type;
developing quality, governance, availability, and security rules for each data type;
updating a database storing the estimates and measurements of properties of the subsurface reservoir;
assessing quality, governance, availability, and security of the estimates and measurements of properties of the subsurface reservoir based on the rules for each data type; and
performing exploration activities based on field properties.

2. The method of claim 1, further comprising incorporating estimates and measurements of properties of the subsurface reservoir into the database and performing quality assurance/quality control review of the estimates and measurements of properties of the subsurface reservoir.

3. The method of claim 1, wherein assessing the quality, governance, availability, and security of the estimates and measurements of properties of the subsurface reservoir comprises assigning a first score representing the quality, a second score representing the governance, a third score representing the availability, and a fourth score representing the security for single data type.

4. The method of claim 3, further comprising averaging all rules for one data type to provide a score for that data type.

5. The method of claim 4, further comprising averaging all data types belonging to an organization to provide a score for that organization.

6. The method of claim 1, wherein developing quality, governance, availability, and security rules for each data type comprises identifying at least one master repository for each data type.

7. A method for assessing the maturity of data, the method comprising:
cataloging types of the data;
developing key performance indicators for each data type;
developing quality, governance, availability, and security rules for each data type;
updating a database storing the data; and
assessing quality, governance, availability, and security of the data based on the rules for each data type.

8. The method of claim 7, further comprising incorporating estimates and measurements of properties of the subsurface reservoir into the database and performing quality assurance/quality control review of the estimates and measurements of properties of the subsurface reservoir.

9. The method of claim 7, wherein assessing the quality, governance, availability, and security of the estimates and measurements of properties of the subsurface reservoir comprises assigning a first score representing the quality, a second score representing the governance, a third score representing the availability, and a fourth score representing the security for single data type.

10. The method of claim 9, further comprising averaging all rules for one data type to provide a score for that data type.

11. The method of claim 10, further comprising averaging all data types belonging to an organization to provide a score for that organization.

12. The method of claim 7, wherein developing quality, governance, availability, and security rules for each data type comprises identifying at least one master repository for each data type.
